(19) United States
(12) Patent Application Publication
(10) Pub. No.: US 2010/0040711 A1
(43) Pub. Date: Feb. 18, 2010

(54) ZINC-CARNOSINE -BASED TREATMENT FOR NON ULCER (FUNCTIONAL ) DYSPEPSIA & IRRITABLE BOWEL SYNDROME IN HUMANS AND OTHER ANIMALS

(76) Inventors: Raymond J. Playford, London (GB); David McLain, Woodstock, GA (US)

Correspondence Address: J. W. SEANOR, DVM, P.O. BOX 516, LEXINGTON, KY, 40588, US

(21) Appl. No.: 12/387,647

(22) Filed: May 4, 2009

Related U.S. Application Data
(60) Provisional application No. 61/126,427, filed on May 2, 2008.

(51) Int. Cl. A61K 38/05; A61P 1/00; A61K 36/48; A61K 36/886

(52) U.S. Cl. 424/744; 514/19; 424/757

(57) ABSTRACT

A method for prophylactically treating irritable bowel syndrome in a mammal through the administration of a zinc carnosine composition like zinc carnosine is provided, although other forms of zinc carnosine or other components exhibiting zinc carnosine activity like other metal carnosine complexes may be used. The effective amount of composition to be used will depend upon such factors as the age and weight of the mammal, and whether treatment of existing IBS symptoms, or prevention of the onset of IBS symptoms is desired. A complex comprising such a zinc carnosine composition for prophylactically treating IBS in a mammal is also provided.

This application claims the benefit of priority in U.S. Provisional Application Ser. No. 61/126,427 filed on May 2, 2008.

### FIELD OF THE INVENTION

This invention relates to a method for using a zinc-carnosine composition to prophylactically reduce the risk of and treat irritable bowel syndrome in mammals, and zinc carnosine compositions for such treatments.

### BACKGROUND OF THE INVENTION

Irritable bowel syndrome (“IBS”), also known as “spastic colon,” “mucous colitis,” “spastic colitis,” “nervous stomach,” or “irritable colon,” is a functional disorder of the digestive system in which the small and large intestine do not work properly. While IBS is not a disease, it can cause a number of painful or otherwise unpleasant symptoms in the abdominal area like cramping, bloating, gas, diarrhea, and constipation. At any given time, 10-20% of the general population suffers from IBS, with woman under age 45 being particularly afflicted. Similarly, symptoms of altered bowel habit, discomfort and or vomiting are often found in dogs and cats presenting to small animal practices. Investigations often show no signs of ulceration and a diagnosis of irritable bowel syndrome made, with marked similarities to the human situation. (Simpson 1998).

In the human or animal body, food is passed from the stomach to the duodenum portion of the small intestines where it is digested, and then to the jejunum and ileum portions of the small intestines where the nutrients (e.g., fatty acids, sugars, amino acids) from the digested food are absorbed. The remaining digesta is then passed along to the large intestine (sometimes called the “bowel” or “gut”) in which fluids from this solid mass of digested food are absorbed. Finally, the leftover, unused portion of the food enters the rectum for subsequent discharge from the body through the anus.

In terms of effect, IBS seems to render the nerves and muscles within the small and large intestine extra-sensitive. For instance, the muscles may contract too much when the person or animal eats, thereby resulting in cramping, bloating or diarrhea during or shortly after the meal. The nerves can also be overly sensitive to the stretching of the large intestine as the digesta passes through it due, for example, to gas. Cramping or pain can result.

The prevalence of IBS within society is demonstrated by the British Society of Gastroenterology Guidelines for the Management of Irritable Bowel Syndrome that comprehensively describes the symptoms, diagnosis, and treatment for this functional disorder. The counterparts within the United States are the American College of Gastroenterology and the American Gastroenterology Association. Besides abdominal pain or discomfort, IBS can result in other non-gastroenterological disorders like lethargy, poor sleep, fibromyolgia, backache, urinary frequency, and dyspareunia. IBS can also lead to substantial reductions in quality of life, including refusals of social invitations, and restrictions upon travel and work. Patients in the U.S. suffering from IBS lost an average of 14.8 workdays per year, compared to 8.7 workdays for the asymptomatic population. See Drossman, D. A., Li, Z., Andruzzi, E., et al. “U.S. Householder Survey of Functional Gastrointestinal Disorders. Prevalence, Sociodemography, and Health Impact”, Dig. Dis. Sci 38: 1569-80 (1993). IBS patients also exhibit greater tendencies for anxiety, hostile feelings, sadness, depression, interpersonal sensitivity, and sleep disturbances when compared with the population as a whole. See Whitehead, W. E., Engel, B. T., and Schuster, M. M., “Irritable Bowel Syndrome Physiological and Psychological Differences Between Diarrhea-Predominant and Constipation Predominant Patients”, Dig. Dis. Sci. 20: 404-13 (1980); Svedlund, J., Sjodin, I., Dotevall, G., et al., “Upper Gastrointestinal and Mental Symptoms in the Irritable Bowel Syndrome,” Scand. J. Gastroenterol., 20: 595-601 (1985); Gamborone, J., Dewsnap, P., Libby, G., et al., “Abnormal Illness Attitudes in Patients with Irritable Bowel Syndrome,” J. Psychosom. Res. 39: 227-30 (1995); Ford, M. J., Miller, P. C., Eastwood, J., et al. “Life Events, Psychiatric Illness, and the Irritable Bowel Syndrome,” Gut 28: 160-65 (1987).

Veterinary practices often find dogs and cats having altered bowel habit, discomfort and/or vomiting with little or no evidence of ulceration or gross inflammation. Working dogs or those breeds with a highly nervous or excitable disposition are particularly at risk (Simpson 1998). These symptoms are generally considered to be in keeping with a diagnosis of irritable bowel syndrome or functional dyspepsia (Simpson 1998).

While IBS and inflammatory bowel disease (“IBD”) are sometimes confused by the general public because of their similar names and because they both have effects upon the bowel, these conditions are, in fact, completely distinct in terms of appearance, causation, treatment, and future prognosis. For instance, the main underlying problem caused by IBS is increased sensitivity of the bowel nerves, while IBD produces gross inflammation that is out of control. The main symptoms of IBS, as described above, are bloating and changes in bowel habits, while the symptoms of IBD are passing blood and mucus, and weight loss. The visual appearance of the intestinal lining of a person suffering from IBS is normal, while the intestinal lining of a person suffering from IBD bleeds and is ulcerated. At a microscopic level, the intestinal lining will appear essentially normal in the case of IBS, while lots of inflammatory cells will be present in the case of IBD. For IBS, the symptoms may come and go, and are treatable by changes in diet and administration of drugs. In the case of IBD, if drugs do not eliminate the symptoms, surgery may be required.

Some people exhibit symptoms of IBS in their youth, while others do not develop symptoms until later in life. Young adult women most typically develop IBS symptoms. This may suggest that infection is a cause of IBS. Indeed, a recent study has demonstrated that patients who suffered from bacterial gastroenteritis are ten times more likely to develop IBS one year later compared with the general population. See Rodriguez, L. A. G. and Ruigomez, A., “Increased Risk of Irritable Bowel Syndrome After Bacterial Gastroenteritis: Cohort Study,” B.M.J. 318: 565-66 (1999). Moreover, although IBS has generally been regarded as a condition where no inflammation is occurring, as opposed to predominantly inflammatory diseases in the small and large intestine such as IBD, Crohn's Disease, and ulcerative colitis, more detailed studies suggest that inflammation may be occurring at the microscopic level in the small and large intestine with increased numbers of lymphocytes. See Tornblom, H., Lindberg, G., Nyberg, B., and Veress, B., “Full-Thickness Biopsy of the Jejunum Reveals Inflammation and Enteric Neuropathy in Irritable Bowel Syndrome,” Gastroenterology 123: 1972-79 (2002). It has also recently been shown that resetting of the nerves in the intestinal wall may occur during intestinal inflammation, particularly when caused by infection. See Stead, R. H., “Nerve Remodeling During Intestinal Inflammation,” Ann. N.Y. Acad. Sci. 664: 443-55 (1992).

Commonly accepted treatment of IBS focuses upon diet changes, medicine, and stress relief. Some foods that have been found to cause IBS symptoms are fatty foods like french fries, milk products like cheese or ice cream, chocolate, alcohol, caffeine found in coffee, tea, and some sodas, and carbonated drinks. Thus, a doctor may advise an IBS patient to reduce or cease altogether the consumption of these food items. At the same time, increased consumption of fiber-intense foods like bran, bread, cereal, beans, fruit, and vegetables can make stools softer, bulkier, and easier to pass, thereby relieving constipation. IBS patients suffering from diarrhea, by contrast will often be advised to reduce fiber consumption.

In both humans and animals, dietary change, by itself, however, will usually be insufficient to treat IBS (Simpson 1988). Therefore, doctors or veterinarians often prescribe medicines like laxatives to treat constipation, antispasmodics to slow contractions in the large intestine to help with diarrhea and pain, and antidepressants to help those IBS patients suffering from severe pain. In animal practice, major sedative drugs such as chlordiazepoxide and dicyclomine may be also used, often for prolonged periods (Simpson 1988) Powerful steroid drugs, often used for inflammatory bowel conditions such as Crohns disease and ulcerative colitis, are ineffective for patients with irritable bowel syndrome. This was also shown in a recent study where administration of predinisolone was found to be ineffective for treating post-infectious IBS. See Dunlap, S. P., Jenkins, D., Neal, K. R., et al., “Randomized, Double-Blind, Placebo-Controlled Trial of Prednisolone in Post-Infectious Irritable Bowel Syndrome,” Ailment Pharmcol. Ther. 18: 77-84 (2003). Moreover, doctors and patients are naturally concerned by long-time use of steroid-based drugs due to the documented side effects. Furthermore, antidepressant medications may be counter-productive for patients with major psychological problems, since their prescription may reinforce abnormal illness behavior and prevent them from effectively addressing underlying psychological problems. Human patients and animals can also become dependent upon antispasmodic, sedative and antidepressant drugs.

Carnosine is a natural component of the body and consists of two joined amino acids (di-peptide) comprising beta-alanine and L-histidine. It is naturally present in long-living cells such as muscle and nerves where, amongst other actions, it probably plays a role in reducing injury by acting as an antioxidant.

Zinc-Carnosine is an artificially produced derivative of carnosine where it is linked in a one-to-one ratio to provide a polymeric structure. Zinc-Carnosine has been extensively studied in animal models of ulceration and gross inflammation and also as a potential anti-ulcer drug in clinical trials. Zinc-Carnosine possesses antioxidant effects (Hirashi H et al 1999) and is able to stimulate growth (proliferation) in a variety of cell lines including human umbilical vein endothelial cells, human foreskin fibroblast cells, and human MCT3-E1 cells (osteoblasts).

Zinc-Carnosine has been shown to have beneficial effects in various animal models of gut ulceration and gross inflammation including water immersion stress and HCl-ethanol gastric damage models (Maksukura & Tanaka, 2000) and in the trinitrobenzene sulphonic acid (TNBS) model of colitis (Yoshikawa T et al. 1997). Clinical trials for the treatment of gastric ulcer have also shown a beneficial effect above placebo (Miyoshi A et al 1992).

However, zinc-carnosine therapy has not previously been applied to IBS human or animal patients within the medical or veterinary community, since there is no role for using anti-ulcer medication to treat the increased sensitivity of the bowel nerves that may cause many of the symptoms of IBS. Prior to the present invention, no scientific or medical tests have shown any efficacy of zinc carnosine for treating IBS. Therefore, the standard treatments for IBS are drug-based.

### SUMMARY OF THE INVENTION

A method for nutritional support to prophylactically reduce the risk or incidence as well as a method of treating IBS in a mammal through the administration of a Zinc-carnosine composition is provided according to the invention. The zinc-carnosine composition preferably is the crystalline form, although other sources of zinc-carnosine, chemically or enzymatically-modified zinc-carnosine, that has been processed to improve or enhance its characteristics or performance, or other components exhibiting zinc-carnosine activity such as other metal carnosine compositions may be used. The effective amount of zinc carnosine to be used will depend upon such factors as the age and weight of the mammal, the bioactivity level of the zinc-carnosine composition, and whether treatment of existing IBS symptoms, or prevention of the onset of IBS symptoms is desired.

A complex comprising a zinc-carnosine composition for prophylactically reducing the risk or incidence as well as treating IBS in a mammal is also provided according to the invention. The zinc-carnosine composition used according to the present invention should preferably comprise about 10 to 400 mg per day of the complex by volume.

### DETAILED DESCRIPTION OF THE PREFERRED EMBODIMENT

Use of various zinc carnosine compositions to prophylactically reduce the risk or incidence as well as to treat IBS is provided by the invention. In one embodiment, the invention may take the form of a method for administering an effective amount of such a zinc-carnosine composition to a patient (be it animal or human) who is suffering from IBS to prophylactically treat the symptoms of such syndrome. Likewise, the invention may take the form of a specific zinc-carnosine composition that is efficacious for the prophylactic treatment of IBS.

An “effective amount” of a zinc-carnosine composition is an amount sufficient to prevent, treat, reduce, and/or ameliorate the symptoms and/or underlying causes of IBS. In some instances, an “effective amount” is sufficient to eliminate the symptoms of IBS and, perhaps, overcome IBS, itself. In the context of the present invention, the terms “treat” and “therapy” and the like refer to alleviate, slow the progression, prophylaxis, attenuation, or cure of existing IBS symptoms. “Prevent,” as used herein, refers to putting off, delaying, slowing, inhibiting, or otherwise stopping, reducing, or ameliorating the onset of such IBS symptoms. “Prophylactic risk reduction and or treatment,” as used herein, means either the administration of the remedy in the absence of IBS symptoms to prevent the onset or occurrence of IBS symptoms within the large or small intestine, or the treatment of IBS symptoms that already exist within the large or small intestine using the remedy.

Several different criteria for diagnosing IBS have been developed within the medical community, and therefore are readily available for such diagnosis of IBS. The Manning criteria call for abdominal pain relieved by defecation, looser stools with onset of pain, more frequent stools with onset of pain, abdominal distension, passage of mucus in stools, or sensation of incomplete evacuation. The Rome I criteria requires at least three months of recurrent symptoms of: (1) abdominal pain or discomfort relieved by defecation, or associated with a change in stool frequency or stool consistency; and (2) two or more of the following symptoms on at least 25% of occasions or days: altered stool frequency, altered stool form, altered stool passage, passage of mucus, and bloating or distension. The Rome II criteria looks for at least 12 weeks over the past 12 months of abdominal discomfort or pain having two of the following three features: (1) relieved by defecation; (2) associated with a change in frequency of stools; and (3) associated with a change in consistency of stool. A patient satisfying any of these IBS criteria who seeks medical attention will have administered by a doctor a physical examination, and possibly also have additional tests such as blood tests, x-rays of the large intestine, or an endoscopy to support the IBS diagnosis. Similarly, animals presenting to the veterinarian may present with multiple symptoms including vomiting, change in bowel habit and abdominal bloating or discomfort. Investigations performed are similar to those in the human. In IBS affecting both humans and animals, no frank ulceration or gross ulceration is present whereas this is expected in patients with inflammatory bowel disease (IBD).

For purposes of this invention, the patient to whom the zinc carnosine composition is to be administered is preferably humans and domesticated animals like dogs, and cats but also includes other mammals such as horses, and livestock like pigs and cows.

The zinc-carnosine composition of the present invention constitutes a component having zinc-carnosine activity. This includes: zinc-carnosine itself; chemically or enzymatically-modified zinc-carnosine, zinc-carnosine that has been processed to change its form or content; zinc-carnosine supplemented with one or more additives to improve or enhance its characteristics or performance; ingredients like lactoferrin, casein, whey that have been extracted from colostrum or milk, or aloe vera, mastic gum, produced from hosts like plants (e.g., tobacco) or bacteria; and food or drink products incorporating such zinc-carnosine or other component having zinc-carnosine activity for ease of administration to a subject.

Zinc-carnosine is commercially available from a number of sources of finished tablets, capsules, gel and powder. Such manufacturers include but are not limited to Hamari Chemicals, Ltd of Osaka, Japan.

A principal advantage of dry, powdered zinc carnosine is that it may be conveniently contained in a capsule or tablet using technology that is readily known in the art. Alternatively, the zinc carnosine may be purchased as a bulk powder that can be added in a measured amount to water, a drink, or food item for easy consumption. Examples of such drinks include milkshakes, protein shakes, drink concentrates, and fruit juices. Examples of such food items include energy bars, candy bars, yoghurt, kefir, cottage cheese, soft cheese, and ice cream. Liquid zinc carnosine may be drunk by itself in a measured amount by the patient, or added in a measured amount to another drink or food item, such as the ones listed above. Formulations to improve taste and appearance has relevance for humans but also having particular relevance for use in domestic animals

Finally, it is envisioned under this invention that the zinc-carnosine composition may be prepackaged in a measured amount in a tablet, capsule, soft gel, powder sachet, drink or food item, so as to administer for convenience the correct dosage of the composition to a patient for nutritional supporting to prophylactically reduce the risk and incidence as well as treating IBS. The possible list of prepackaged drink or food items includes, but is not limited to, those mentioned above.

One or more additives may advantageously be added to the zinc-carnosine composition of the present invention to improve or enhance its characteristics or performance. For example, a flavoring agent like licorice, chocolate, strawberry, or lemon in the case of humans and beef, chicken or fish can be added in the case of animals to the zinc-carnsoine product to make it more palatable to swallow. Some functional ingredients (i.e. Licorice) have the added benefit of changing the contractions of the intestines that can otherwise lead to diarrhea or constipation, and reducing the amount of acid produced in the stomach.

Various minerals such as calcium, magnesium and selenium as well as vitamins, such as Vitamin E, may also be added as an ingredient to the tablet, capsule, liquid or powdered zinc carnosine composition using techniques known in the industry. Such components will assist in the general digestive health of the subject as the subject may be deficient in some of these compositions if suffering from prolonged diarrhea or vomiting.

For purposes of this invention, the zinc-carnosine composition may be administered to a patient as a prophylactic treatment for IBS in any of the forms discussed above. In the case of a human averaging 50 kg or more in weight, the zinc-carnosine composition should be administered in a single or multiple daily doses amounting in total from about 5 to about 200 mg/day, more preferably about 15-150 mg/day, even more preferably about 75-150 mg/day.

The zinc-carnosine composition may also be effective for preventing the onset or reducing the incidence of IBS when taken on a regular basis over time. This could be a daily, in one or divided doses or taken on an intermittent basis. For example, a human averaging 50 kg or more in weight who is not suffering yet from IBS symptoms should take single or multiple doses amounting in total to about 5-200 mg/day, more preferably about 30-150 mg/day, even more preferably about 75-150 mg/day, as a preventive treatment against the onset of IBS symptoms.

For children younger than age 12 and or people less than 50 kg of weight, the dosages discussed above for treating or preventing IBS may need to be cut in half, at the discretion of the health care provider. The dosages may also be adjusted to take into account the weight of the patient, as is well known in the medical arts. This is particularly important for administering the zinc-carnosine composition to patients that are not humans. With regards to the use for dogs and cats in veterinary practice, the preferred dosing to be used is from about 10 to about 60 mg/day, even more preferably from about 30 to about 60 mg/day.

While not wanting to be bound by any particular theory, it is plausible that one reason why patients develop IBS may be that an infection within the small or large intestine causes continuing microscopic inflammation that results in changes in the enteric nervous system, which, in turn, increases the sensitivity of the small or large intestine. Factors that have immune-modulatory activity may, therefore, influence the natural history and severity of IBS acting through pathways such as the enteric nervous system, as well as gut flora.

The present invention confirms that zinc-carnosine can be used to achieve an immune response in the intestine that will dampen down the excessive response caused by the inflammatory cells that are fighting against each other and against the nerves in the intestinal walls thereby reducing the local microscopic inflammation. In turn, the present invention demonstrates that zinc carnosine can be successfully used to prophylactically treat IBS. Zinc supplementation has previously been shown to influence a mixed lymphocyte cell response, however, the present invention demonstrates that zinc-carnosine has a more beneficial effect. In addition, the rationale of the previous study for studying zinc in mixed lymphocyte response was related to organ rejection and not related to its potential value in gut nerve sensitivity of irritable bowel syndrome.

### EXAMPLE 1

Ellie, a three year old, sixteen pound West Highland Terrier presented to vet with visible discomfort, loose stools and intermittent diarrhea. Intestinations showed no evidence of ulceration or gross inflammation and stool sample showed no parasites were present. The diagnosis of functional dyspepsia or irritable bowel syndrome made. Dog was given zinc-carnosine at 30 mg/day and showed significant improvement in the two week and four week follow-up visits.

### EXAMPLE 2

Katelyn, a six year old, fourteen pound Boston Terrier presented to vet with visible discomfort, diarrhea, vomiting and inappetence. Intestinations and endoscopy showed no evidence of ulceration or gross inflammation. Blood tests indicated no abnormalities and a stool samples indicated the dog was parasite free. The diagnosis of functional dyspepsia or irritable bowel syndrome made. Dog was given zinc-carnosine at 30 mg/day and conditions improved on a daily basis. No further symptoms were reported on subject on a four week check up.

### EXAMPLE 3

A five year old, eight pound Siamese cat presented to vet with severe diarrhea, severe visible discomfort, inappetence and losing weight. Intestinations showed no evidence of ulceration or gross inflammation. Blood tests indicated no abnormalities and a stool samples indicated the cat was parasite free. The diagnosis of functional dyspepsia or irritable bowel syndrome made. Cat was given zinc-carnosine at 15 mg BID and conditions improved significantly within seven days. No further symptoms on a four week check up.

The successful use of zinc-carnosine to treat IBS is interesting, because, since inflammation abnormal nervous innervations and interactions between the two have not been generally been recognized as part of the pathophysiology of the IBS functional disorder, zinc carnosine has not previously been deemed to be a relevant or appropriate treatment or preventive remedy for IBS within the medical community.

At the same time, zinc-carnosine is based on a natural product which, based on current evidence, appears to have little or no side effects. It therefore can be taken long-term as a preventive agent, unlike many of the drug treatments currently used for IBS.

The above specification, examples and data provide a complete description of the invention relating to the method of prophylactic treatment of IBS, and the composition for such prophylactic treatment. Since many embodiments of the invention can be made without departing from the spirit and scope of the invention, the invention resides in the claims hereinafter appended.

What is claimed:

1. A method for providing nutritional support, prophylactic risk reduction and incidence as well as treatment of irritable bowel syndrome in a mammal comprising administering to the mammal an effective amount of a composition comprising zinc-carnosine composition.

2. The method according to claim 1, wherein the zinc-carnosine composition comprises zinc-L-carnosine.

3. The method according to claim 2, wherein the zinc-L-carnosine is obtained by a chemical manufacturing method comprising chelating elemental zinc and L-carnosine.

4. The method according to claim 1, wherein the zinc-carnosine composition has sufficient bioactivity, such that when it is added to a cell culture medium at a final concentration of 1 mg protein/ml, its ability to stimulate growth (proliferation) of intestinal cells is at least 40% of the result seen when pure epidermal growth factor has been added to the same system at a final concentration of 10 μg/ml, and its ability to stimulate movement (restitution) of these cells is at least 40% of the result seen when pure epidermal growth factor has been added to the same system at a final concentration of 10 μg/ml.

5. The method according to claim 1, wherein the effective amount is from about 5 to about 400 mg of zinc-carnosine and the composition is administered one or more times daily.

6. The method according to claim 1, wherein the effective amount is from about 5 to about 400 mg of zinc-carnosine administered at least once daily for the treatment of a mammal suffering from IBS symptoms.

7. The method according to claim 1, wherein the effective amount is from about 5 to about 400 mg of zinc-carnosine administered at least once daily for the prevention of the onset of IBS symptoms in a mammal

8. The method according to claim 1, wherein the zinc-carnosine composition comprises zinc carnosine is in dry crystalline form.

9. The method according to claim 8, wherein about 5 to about 400 mg of the zinc-carnosine composition is administered at least once daily.

10. The method according to claim 9, wherein from about 5 to about 400 mg of the zinc-carnosine composition is administered at least once daily for the treatment of a mammal suffering from IBS symptoms.

11. The method according to claim 9, wherein from about 5 to about 400 mg of zinc-carnosine is administered daily for the prevention of the onset of IBS symptoms in a mammal.

12. The method according to claim 1, wherein the zinc-carnosine composition is administered by an oral route.

13. The method according to claim 13, wherein the zinc-carnosine composition is administered in the form of a capsule, tablet, liquid, food product, or drink product.

14. The method according to claim 1, wherein the zinc-carnosine composition further comprises or is added to other minerals or vitamins.

15. A method of prophylactically treating irritable bowel syndrome in a mammal comprising administering a composition comprised of zinc-carnosine in an amount effective to reduce the symptoms of IBS, or prevent the onset of the IBS symptoms.

16. The method according to claim 15, wherein the zinc-carnosine composition comprises from about 5 to about 400 mg of zinc-carnosine.

17. The method according to claim 15, wherein the effective amount of zinc-carnosine to be administered daily for the treatment of the mammal suffering from IBS symptoms is from about 1 to about 100 g.

18. The method according to claim 15, wherein the effective amount of zinc-carnosine administered at least once daily for the prevention of the onset of IBS symptoms in the mammal is from about 5 to about 400 mg.

19. The method according to claim 1, wherein the zinc-carnosine composition comprises zinc-carnosine in a liquid dose form.

20. The method according to claim 19, wherein the effective amount of zinc-carnosine in liquid dose form is from about 5 to about 400 mg administered at least once daily.

21. The method according to claim 19, wherein the effective amount of zinc-carnosine in a liquid form to be ingested daily for the treatment of the mammal suffering from IBS symptoms is 5 to 400 mg.

22. The method according to claim 19, wherein the effective amount of zinc-carnosine in liquid dose form to be administered at least once daily for the prevention of the onset of IBS symptoms in the mammal is from about 5 to about 400 mg.

23. The method according to claim 19, wherein the zinc-carnosine composition is administered by an oral route.

24. The method according to claim 23, wherein the zinc-carnosine composition is administered in the form of a capsule, tablet, liquid, food product, or drink product.

25. The method according to claim 23, wherein the zinc-carnosine composition further comprises a flavoring agent.

26. The method according to claim 23, wherein the zinc-carnosine composition further comprises minerals and vitamins.

27. A composition for the prophylactic risk reduction and or treatment of irritable bowel syndrome in a mammal comprising an effective amount of zinc-carnosine.

28. The composition according to claim 27, wherein the composition comprises zinc-carnosine is in liquid dose form.

29. The composition according to claim 27, wherein the zinc-carnosine composition further comprises a flavoring agent.

30. The composition according to claim 27, wherein the flavoring agent also has a functional benefit.

31. The composition according to claim 30 wherein the flavoring agent is selected from the group consisting of licorice, Vitamin E, aloe vera and combinations thereof.

32. The composition according to claim 27, wherein the zinc-carnosine composition further comprises a coloring agent.

33. The complex composition according to claim 27, wherein the composition further comprises a mineral or a vitamin.
